(19) United States
(12) Patent Application Publication
(10) Pub. No.: US 2008/0269890 A1
(43) Pub. Date: Oct. 30, 2008

(54) INTRAOCULAR LENS WITH PERIPHERAL REGION DESIGNED TO REDUCE NEGATIVE DYSPHOTOPSIA

(75) Inventors: Michael J. Simpson, Arlington, TX (US); Xiaoxiao Zhang, Fort Worth, TX (US)

Correspondence Address: ALCON, IP LEGAL, TB4-8, 6201 SOUTH FREEWAY, FORT WORTH, TX, 76134, US

(73) Assignee: ALCON UNIVERSAL LTD., Fort Worth, TX (US)

(21) Appl. No.: 11/742,041

(22) Filed: Apr. 30, 2007

(51) Int. Cl. A61F 2/16

(52) U.S. Cl. 623/6.46

(57) ABSTRACT

In one aspect, the invention provides an intraocular lens (IOL) that includes an optic and a peripheral optical flange that surrounds the optic. The optic can form an image of a field of view on the IOL user's retina and the peripheral flange can inhibit dysphotopsia. By way of example, the peripheral flange can include at least one textured surface that is adapted to receive peripheral light rays entering the eye at large visual angles so as to cause their scattering in order to inhibit dysphotopsia, e.g., by preventing the formation of a secondary peripheral image or scattering some light to a shadow region between such a secondary image and an image formed by the IOL.

### RELATED APPLICATIONS

This application is related to the following patent application that are concurrently filed herewith, each of which is incorporated herein by reference: “Intraocular Lens with Asymmetric Optics” (Attorney Docket No. 3360), “IOL peripheral Surface Designs to Reduce Negative Dysphotopsia” (Attorney Docket No. 3345), “Intraocular Lens with Asymmetric Haptics” (Attorney Docket No. 3227), “Intraocular Lens With Edge Modification,” (Attorney Docket No. 3225), “A New Ocular Implant to Correct Dysphotopsia, Glare, Halo, and Dark Shadow” (Attorney Docket No. 3226), “Haptic Junction Designs to Reduce Negative Dysphotopsia,” (Attorney Docket No. 3344), and “Graduated Blue Filtering Intraocular Lens,” (Attorney Docket No. 2962).

### BACKGROUND

The present invention relates generally to intraocular lenses (IOLs), and particularly to IOLs that provide a patient with an image of a field of view without the perception of visual artifacts in the peripheral visual field.

The optical power of the eye is determined by the optical power of the cornea and that of the natural crystalline lens, with the lens providing about a third of the eye's total optical power. The process of aging as well as certain diseases, such as diabetes, can cause clouding of the natural lens, a condition commonly known as cataract, which can adversely affect a patient's vision.

Intraocular lenses are routinely employed to replace such a clouded natural lens. Although such IOLs can substantially restore the quality of a patient's vision, some patients with implanted IOLs report aberrant optical phenomena, such as halos, glare or dark regions in their vision. These aberrations are often referred to as “dysphotopsia.” In particular, some patients report the perception of dark shadows, particularly in their temporal peripheral visual fields. This phenomenon is generally referred to as “negative dysphotopsia.”

Accordingly, there is a need for enhanced IOLs, especially IOLs that can reduce dysphotopsia, in general, and the perception of dark shadows or negative dysphotopsia, in particular.

### SUMMARY

The present invention generally provides intraocular lenses (IOLs) in which the peripheral region of the optic is designed to alleviate, and preferably eliminate, the perception of shadows that some IOL patients report.

The present invention is based, in part, on the discovery that the shadows perceived by IOL patients can be caused by a double imaging effect when light enters the eye at very large visual angles. More specifically, it has been discovered that in many conventional IOLs, most of the light entering the eye is focused by both the cornea and the IOL onto the retina, but some of the peripheral light misses the IOL and it is hence focused only by the cornea. This leads to the formation of a second peripheral image. Although this image can be valuable since it extends the peripheral visual field, in some IOL users it can result in the perception of a shadow-like phenomenon that can be distracting.

To reduce the potential complications of cataract surgery, designers of modern IOLs have sought to make the optical component (the “optic”) smaller (and preferably foldable) so that it can be inserted into the capsular bag with greater ease following the removal of the patient's natural crystalline lens. The reduced lens diameter, and foldable lens materials, are important factors in the success of modern IOL surgery, since they reduce the size of the corneal incision that is required. This in turn results in a reduction in corneal aberrations from the surgical incision, since often no suturing is required. The use of self-sealing incisions results in rapid rehabilitation and further reductions in induced aberrations. However, a consequence of the optic diameter choice is that the IOL optic may not always be large enough (or may be too far displaced from the iris) to receive all of the light entering the eye.

Moreover, the use of enhanced polymeric materials and other advances in IOL technology have led to a substantial reduction in capsular opacification, which has historically occurred after the implantation of an IOL in the eye, e.g., due to cell growth. Surgical techniques have also improved along with the lens designs, and biological material that used to affect light near the edge of an IOL, and in the region surrounding the IOL, no longer does so. These improvements have resulted in a better peripheral vision, as well as a better foveal vision, for the IOL users. Though a peripheral image is not seen as sharply as a central (axial) image, peripheral vision can be very valuable. For example, peripheral vision can alert IOL users to the presence of an object in their field of view, in response to which they can turn to obtain a sharper image of the object. It is interesting to note in this regard that the retina is a highly curved optical sensor, and hence can potentially provide better off-axis detection capabilities than comparable flat photosensors. In fact, though not widely appreciated, peripheral retinal sensors for visual angles greater than about 60 degrees are located in the anterior portion of the eye, and are generally oriented toward the rear of the eye. In some IOL users, however, the enhanced peripheral vision can lead to, or exacerbate, the perception of peripheral visual artifacts, e.g., in the form of shadows.

Dysphotopsia (or negative dysphotopsia) is often observed by patients in only a portion of their field of vision because the nose, cheek and brow block most high angle peripheral light rays—except those entering the eye from the temporal direction. Moreover, because the IOL is typically designed to be affixed by haptics to the interior of the capsular bag, errors in fixation or any asymmetry in the bag itself can exacerbate the problem—especially if the misalignment causes more peripheral temporal light to bypass the IOL optic.

In many embodiments, an IOL of the invention is configured so as to capture or redirect peripheral light rays entering the eye in a manner that would inhibit dysphotopsia. By way of example, in some embodiments, an IOL of the invention can include an optic surrounded by a peripheral flange that is adapted to receive light rays entering the eye at large visual angles. In some embodiments, such a flange can scatter the incident light rays (e.g., via one or more textured surfaces) so as to inhibit dysphotopsia, e.g., by inhibiting the formation of a separate peripheral image from that formed by the optic, or by directing some light into a reduced intensity (shadow) region between a second peripheral image, formed by light rays entering the eye that miss the IOL, and a primary image formed by the optic. In other embodiments, the flange can be opaque so as to inhibit the incident peripheral light rays from reaching the retina, or to reduce the intensity of such rays so as to attenuate a secondary peripheral image that might be formed on the retina by some light rays entering the eye that miss the IOL. In yet other embodiments, the IOL can include an optic that is sufficiently large to inhibit peripheral light rays from forming a secondary image, e.g., via scattering or absorption, or by focusing those rays such that a single image of a field of view is formed.

In one aspect, the invention provides an intraocular lens (IOL) that includes an optic and a peripheral optical flange surrounding that optic. The optic forms an image of a field of view on the retina of a patient's eye in which the IOL is implanted and the peripheral flange inhibits the perception of visual artifacts (e.g., dysphotopsia) in the patient's peripheral visual field. By way of example, in some cases, the peripheral flange captures peripheral light rays entering the eye at large visual angles and inhibits those rays from forming a secondary peripheral image, and in other cases, the peripheral flange directs some light (e.g., by scattering) to a shadow region between such a secondary image and an image formed by the IOL. In many cases, the optic has a diameter in a range of about 4 millimeters (mm) to about 9 mm and the peripheral flange has a width in a range of about 0.5 mm to about 1 mm.

In a related aspect, the peripheral flange includes at least one textured surface, e.g., an anterior textured surface, that is adapted to cause scattering of light incident thereon so as to inhibit dysphotopsia. For example, the textured surface can receive peripheral light rays entering the eye at large visual angles (e.g., at angles in a range of about 50 to about 80 degrees) and to cause scattering of those rays so as to inhibit them from forming a secondary image, which would otherwise cause dysphotopsia. Alternatively, the textured surface can direct at least some of the light rays incident thereon to the shadow region. The texturing of the surface can be achieved, for example, via a plurality of surface undulations having amplitudes that create an optical path distance effect of the order of visible light wavelengths. For example, in some embodiments the physical surface amplitudes can range from about 0.2 microns to about 2 microns. Alternatively, the textured peripheral flange can scatter at least some of the light rays incident thereon into a shadow region between a secondary peripheral image and an image formed by the IOL's optic.

In another aspect, the peripheral optical flange is opaque to visible radiation. In some cases, such an opaque peripheral flange can receive peripheral light rays entering the eye at large visual angles and can inhibit them (e.g., via absorption) from forming a secondary retinal image. Alternatively, the opaque peripheral flange can attenuate the intensity of peripheral light rays passing therethrough.

In another aspect, the peripheral flange is translucent to visible radiation. Some of the light rays that are incident on the translucent flange (e.g., light rays entering the eye at large visual angles) may pass through the flange, but diffusely. This can inhibit the formation of a secondary peripheral image and/or can direct sufficient light into the shadow region to inhibit the perception of visual artifacts in the peripheral visual field.

In another aspect, the peripheral flange can include a diffractive structure disposed on a surface thereof (e.g., disposed on an anterior surface of the flange) that is adapted to direct some of the light incident thereon onto a shadow region between a secondary peripheral image and an image formed by the optic. In some cases, the optical power associated with the diffractive structure is less than optical power of the eye's cornea alone and/or less than the combined optical power of the cornea and that of the optic (e.g., by a factor in a range of about 25% to about 75%).

In yet another aspect, the peripheral flange can include a Fresnel lens for directing the light incident thereon to the retinal shadow region between an image formed by the optic and a second peripheral image formed by rays entering the eye that miss the IOL. In some embodiments, the optical power of the Fresnel lens can be less than the optical power of the eye's cornea alone and/or less than the combined optical power of the cornea and that of the optic (e.g., by a factor in a range of about 25% to about 75%). For example, in some implementations, the optical power of the Fresnel lens is about one-half of the combined optical power of the cornea and that of the IOL's central optic.

In another aspect, in the above IOL, the optic can provide multiple foci. For example, the optic can comprise an anterior surface and a posterior surface, and a diffractive structure disposed on at least one of those surfaces. The diffractive structure can provide a far-focus as well as a near-focus optical power (e.g., a near-focus power in a range of about 1 D to about 4 D).

In another aspect, an IOL is disclosed that includes an optic comprising an anterior surface and a posterior surface, wherein the optic includes a central portion for generating an image of a field of view and a peripheral portion for inhibiting dysphotopsia, e.g., by inhibiting the formation of a secondary peripheral image. By way of example, the optic can have a diameter in a range of about 4 mm to about 9 mm, with its central portion having a diameter in a range of about 3.5 mm to about 8 mm and its peripheral portion having a width in a range of about 0.5 mm to about 1 mm.

In a related aspect, in the above IOL, the peripheral portion of the optic includes a textured region (e.g., characterized by a plurality of surface undulations) that is adapted to scatter light rays incident thereon (e.g., the peripheral light rays entering the eye at large visual angles) so as to inhibit dysphotopsia, e.g., by inhibiting the formation of a secondary retinal image or by directing some light into the shadow region. While the textured region can be disposed on the anterior or the posterior surface, more preferably, it is disposed on the peripheral portion of the anterior surface.

In other aspects, the optic's peripheral portion can be opaque or translucent. The opaque peripheral portion can inhibit peripheral light rays entering the eye at large visual angles from forming a secondary image that would cause dysphotopsia, for example, via absorption or diffusion of those rays. Alternatively, the opaque portion can cause a substantial reduction in the intensity of such a secondary image. The translucent portion can inhibit dysphotopsia by inhibiting (ameliorating or preventing) the formation of a secondary peripheral image and/or by directing at least some of the light incident thereon, e.g., via diffusion, into the shadow region.

In another aspect, a diffractive structure can be disposed on the optic's peripheral portion to direct some light to a shadow region between a secondary peripheral image and an image formed by the IOL. By way of example, the diffractive structure can provide a focusing power less than that of the cornea alone and/or less than the combined power of the cornea and the IOL.

In yet another aspect, a Fresnel lens can be disposed on the peripheral portion of an anterior and/or posterior surface of the optic so as to direct light to a shadow region between an image formed by the IOL and a secondary peripheral image formed by light rays entering the eye that miss the IOL.

In another aspect, an IOL is disclosed having focusing surfaces that are sufficiently large so as to focus not only axial rays entering the eye but also rays entering the eye at large visual angles to form a single image of a field of view. By way of example, such an IOL can include an optic having an anterior surface and a posterior surface disposed about an optical axis, where the surfaces have a diameter greater than about 6.5 mm (e.g., in a range of about 6.5 mm to bout 9 mm).

In yet another aspect, a diffractive structure can be disposed on at least one of the IOL's anterior and/or posterior surfaces such that the IOL would be capable of providing not only a far-focus power but also a near-focus power (e.g., corresponding to an add power in a range of about 1 D to about 4 D).

In other aspect, a method of correcting vision is disclosed that includes providing an IOL having a central optic and a peripheral flange that surrounds that optic, and implanting the IOL in a patient's eye. The optic is adapted to form an image of a field of view and the flange is adapted to inhibit dysphotosia.

In another aspect, the invention provides a method of inhibiting dysphotopsia in a visual field of a patient's eye in which an IOL is implanted by ensuring that the IOL is sufficiently large so as to capture peripheral light rays entering the eye at large visual angles or to direct those rays to the retina so as to form a single image of a field of view.

Further understanding of the invention can be obtained by reference to the following detailed description, in conjunction with the associated drawings, which are briefly discussed below.

### BRIEF DESCRIPTION OF THE DRAWINGS

FIG. 1A is a schematic top view of an IOL according to one embodiment of the invention,

FIG. 1B is a schematic side view of the IOL depicted in FIG. 1A,

FIG. 1C schematically depicts an IOL according to another embodiment that includes a central optic and a peripheral flange, where the flange is slanted relative to the central optic,

FIG. 2A schematically shows a conventional IOL implanted in a patient's eye, illustrating schematically the formation of a secondary image by peripheral light rays that enter the eye at large visual angles and miss the IOL,

FIG. 2B schematically shows an IOL according to one embodiment of the invention implanted in a patient's eye, illustrating schematically that the IOL's peripheral flange inhibits the formation of a secondary image by peripheral light rays entering the eye at large visual angles,

FIG. 2C schematically shows an IOL according to one embodiment of the invention implanted in a patient's eye, illustrating that the IOL's textured peripheral flange causes scattering of some light rays into a shadow region between an image formed by the IOL's optic and second peripheral image formed by rays entering the eye that miss the IOL,

FIG. 3 is schematic anterior view of an IOL according to another embodiment of the invention,

FIG. 4 is a schematic side view of an IOL according to another embodiment of the invention,

FIG. 5A is a schematic side view of an IOL according to another embodiment of the invention,

FIG. 5B is a schematic side view of an IOL according to another embodiment of the invention that includes an optic surrounded by a focusing flange,

FIG. 5C is a schematic side view of an IOL according to another embodiment of the invention having a diffractive peripheral flanges,

FIG. 5D is a schematic anterior view of the IOL of FIG. 5C,

FIG. 5E is a schematic side view of an IOL according to another embodiment of the invention having a Fresnel lens on an anterior surface of its peripheral flanges,

FIG. 6A is a schematic side view of an IOL according to another embodiment of the invention,

FIG. 6B is a schematic anterior view of the IOL of FIG. 6A,

FIG. 7A schematically depicts the IOL of FIG. 6A implanted in a patient's eye, illustrating schematically that the IOL's peripheral portion inhibits dysphotopsia,

FIG. 7B schematically depicts one exemplary implementation of the IOL of FIG. 6A implanted in a patient's eye, where the IOL's peripheral textured portion cause scattering of some light rays into a shadow region between an image formed by the IOL and a secondary peripheral image formed by light rays entering the eye that miss the IOL,

FIG. 8A is a schematic side view of an IOL according to another embodiment of the invention having an opaque peripheral portion,

FIG. 8B is a schematic anterior view of the IOL of FIG. 8B,

FIG. 9 is a schematic side view of an IOL according to another embodiment of the invention,

FIG. 10A is a schematic side view of an IOL according to another embodiment of the invention,

FIG. 10B is a schematic side view of an IOL according to another embodiment of the invention having a Fresnel lens disposed on a peripheral portion of its anterior surface,

FIG. 11 is a schematic side view of an IOL according to another embodiment of the invention,

FIG. 12 schematically depicts the IOL of FIG. 11 implanted in a patient's eye, illustrating schematically that the IOL inhibits dysphotopsia,

FIG. 13A is a multifocal IOL according to another embodiment of the invention having a diffractive structure on an anterior surface thereof,

FIG. 13B is a schematic anterior view of the IOL of FIG. 12A.

### DETAILED DESCRIPTION

The present invention generally provides intraocular lenses (IOLs) that ameliorate, and preferably prevent, the perception of dark shadows that some IOL patients report. Such an effect is known generally in the art as dysphotopsia. As discussed in more detail below, in many embodiments, the IOLs of the invention include a central optic that is surrounded by a peripheral flange, where the flange inhibits dysphotopsia, e.g., by inhibiting the formation of a secondary peripheral image or directing some light to a shadow region between such a secondary peripheral image and a primary image formed by the IOL. To this end, in some cases, the peripheral flange can cause scattering of peripheral light rays entering the eye, e.g., at large visual angles, while in other cases, the peripheral flange can be substantially opaque to visible radiation. In yet other cases, the peripheral flange can function as a focusing element by refracting and/or diffracting the peripheral light rays towards a portion of the retina on which the central optic forms an image, or by focusing some light into the shadow region, thus inhibiting dysphotopsia. In other embodiments, rather than utilizing a separate optical flange, the IOL's optic is sufficiently large so as to capture or redirect peripheral light rays entering the eye at large visual angles so as to inhibit dysphotopsia. The term “intraocular lens” and its abbreviation “IOL” are used herein interchangeably to describe lenses that are implanted into the interior of the eye to either replace the eye's natural lens or to otherwise augment vision regardless of whether or not the natural lens is removed.

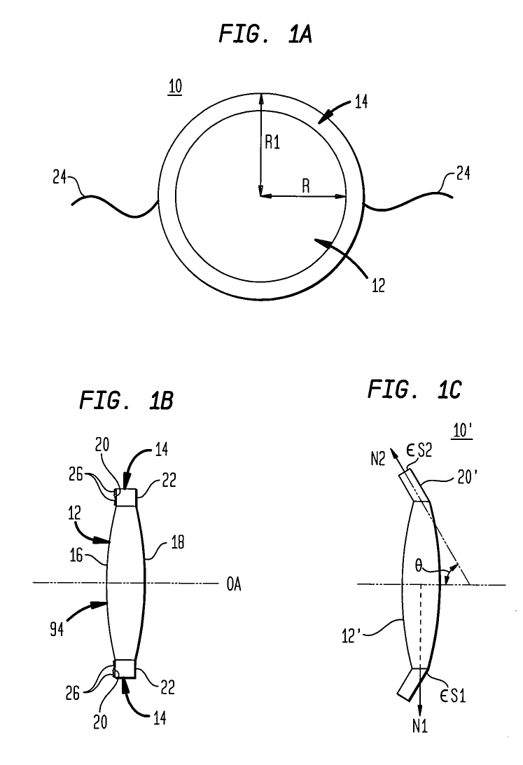

FIGS. 1A and 1B schematically depict an IOL 10 according to one embodiment of the invention that includes a central optic 12 and a peripheral flange 14 disposed about an optical axis OA, where the flange surrounds the central optic. In this embodiment, the central optic has a radius (R) relative to the optical axis in a range of about 2 mm to about 3.5 mm, and the flange has a radius (R′) relative to the optical axis in a range of about 2.5 mm to about 4.5 mm.

The central optic 12 includes an anterior surface 16 and a posterior surface 18 that cooperatively provide a desired optical power. Although in this embodiment the central optic has a bi-convex shape, in other embodiments it can have other shapes, such as convex-concave, plano-convex or plano-concave. Similarly, the peripheral flange includes an anterior surface 20 and a posterior surface 22. Although in this embodiment the anterior and posterior surfaces of the flange are substantially flat, in other embodiments they can be curved to provide focusing of light incident thereon.

The optic 12 and the peripheral flange 14 are preferably formed of a biocompatible material, such as soft acrylic, silicone, hydrogel, or other biocompatible polymeric materials having a requisite index of refraction for a particular application. For example, in some embodiments, they can be formed of a cross-linked copolymer of 2-phenylethyl acrylate and 2-phenyltheyl methacrylate, which is commonly known as Acrysof®. The IOL 10 has also a pair of fixation members (haptics) 24 that facilitate its placement in the eye. The haptics 24 can also be formed of a suitable biocompatible material, such as polymethylmethacrylate. While in some embodiments, the haptics can be formed integrally with the optic, in other embodiments (commonly referred to as multipiece IOLs) the haptics are formed separately and attached to the optic in a manner known in the art. In the latter case, the material from which the haptics are formed can be the same as, or different from, the material forming the optic. It should be appreciated that various haptic designs for maintaining lens stability and centration are known in the art, including, for example, C-loops, J-loops, and plate-shaped haptic designs. The present invention is readily employed with any of these haptic designs.

With continued reference to FIGS. 1A and 1B, the anterior flange surface 20 is textured to cause scattering of light incident thereon. As discussed further below, in this embodiment, once the IOL is implanted in the eye, at least some peripheral light rays entering the eye at large visual angles are incident on the textured anterior flange surface, which causes scattering of those rays so as to inhibit formation of a secondary image. The term “large visual angles,” as used herein, refers to angles relative to the eye's visual axis that are greater than about 50 degrees, e.g., in a range of about 50 to about 80 degrees. In this embodiment, the texturing of the anterior flange surface is achieved by a plurality of surface undulations 26 with physical surface amplitudes that are in a range of about 0.2 microns to about 2 microns. In many cases, the scattering of the light by the textured surface can distribute at least 40 percent, or at least about 90 percent, or at least about 95 percent, of the light incident on the surface randomly over a plurality of directions.

In some implementations, the IOL's peripheral flange can be slanted anteriorly or posteriorly relative to its central optic. By way of example, with reference to FIG. 1C, an IOL 10′ can include a central optic 12′ that is surrounded by a peripheral flange 20′, which is slanted relative to the central optic. More particularly, a normal N1 to an edge surface ES1 of the central optic is substantially orthogonal to an optical axis OA of the IOL, whereas a normal N2 to an edge surface ES2 of the flange forms an angle θ relative to the optical axis. The flange can be configured to inhibit dysphotopsia, e.g., in a manner discussed above and further below. Moreover, in some implementations of this and other embodiments, the thickness of the flange can be less than the minimum (or the average) thickness of the central optic (e.g., by a factor of about 5).

During cataract surgery, a clouded natural lens can be removed and replaced with the IOL 10. By way of example, an incision can be made in the cornea, e.g., via a diamond blade, to allow other instruments to enter the eye. Subsequently, the anterior lens capsule can be accessed via that incision to be cut in a circular fashion and removed from the eye. A probe can then be inserted through the corneal incision to break up the natural lens via ultrasound, and the lens fragments can be aspirated. An injector can be employed to place the IOL, while in a folded state, in the original lens capsule. Upon insertion, the IOL can unfold and its haptics can anchor it within the capsular bag.

In some cases, the IOL is implanted into the eye by utilizing an injector system rather than employing forceps insertion. For example, an injection handpiece having a nozzle adapted for insertion through a small incision into the eye can be used. The IOL can be pushed through the nozzle bore to be delivered to the capsular bag in a folded, twisted, or otherwise compressed state. The use of such an injector system can be advantageous as it allows implanting the IOL through a small incision into the eye, and further minimizes the handling of the IOL by the medical professional. By way of example, U.S. Pat. No. 7,156,854 entitled “Lens Delivery System,” which is herein incorporated by reference, discloses an IOL injector system. The IOLs according to various embodiments of the invention, such as the IOL 10, are preferably designed to inhibit dysphotopsia while ensuring that their shapes and sizes allow them to be inserted into the eye via the injector systems through small incisions.

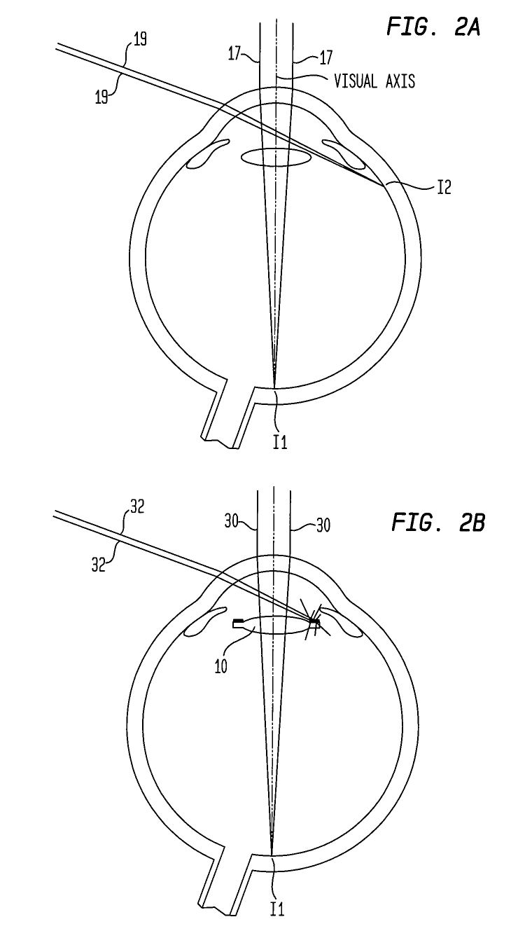

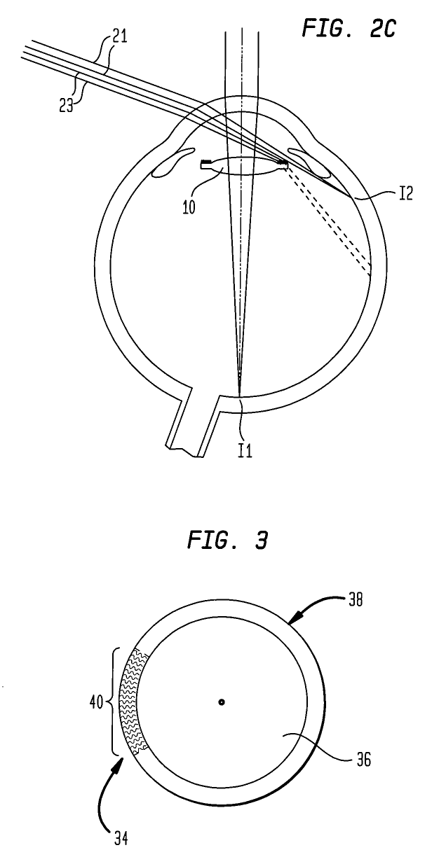

Once implanted in the eye, in this exemplary embodiment, the central optic of the IOL forms an image of a field of view while the IOL's peripheral flange inhibits formation of a secondary peripheral image that would cause dysphotopsia. To further illustrate the role of the peripheral flange in inhibiting dysphotosia, FIG. 2A shows a conventional IOL 28 implanted in the eye and FIG. 2B shows the above IOL 10 implanted in the eye. With reference to FIG. 2A, the conventional IOL 28 can form an image I1 of a field of view by focusing a plurality of light rays (such as rays 17) entering the eye onto the retina. However, a plurality of peripheral light rays (such as rays 19) that enter the eye at large visual angles are refracted by the cornea but miss the IOL 28. As such, those peripheral rays reach the retina at a location separated from the image I1 to form in many cases a secondary peripheral image I2. The formation of such a secondary image can result in the perception of a shadow-like phenomenon between those images by the patient, e.g., in a range of about 25% to about 100%.

In contrast, as shown schematically in FIG. 2B, while the central optic 12 of the IOL 10 forms an image I1 on the patient's retina by focusing a plurality of light rays (such as rays 30) onto the retina, the peripheral light rays (such as light rays 32) entering the eye at large visual angles are incident on the textured anterior surface 20 of the peripheral flange 14. The textured surface causes scattering of the incident peripheral rays, thereby inhibiting them from forming a secondary image on the patient's retina. In this manner, it inhibits dysphotopsia.

In this embodiment, the posterior surface 22 of the flange 14 is not textured (the flange's posterior surface has a smooth surface profile) so as to minimize the potential of posterior capsular opacification (PCO)—though in other embodiments both the posterior surface of the flange or both of its anterior and posterior surfaces can be textured.

In some other implementations of this embodiment, rather than inhibiting the formation of a second peripheral image, the textured flange scatters some light into a shadow region between such a secondary peripheral image and a primary image formed by the IOL so as to inhibit the perception of peripheral visual artifacts, e.g., in the form of dark shadows, by the IOL user while preserving the secondary peripheral image that can be beneficial for peripheral vision. For example, as shown schematically in FIG. 2C, once the IOL 10 is implanted in a patient's eye, its central optic can form an image I1 of a field of view. In this case, however, the IOL is not large enough such that the flange would be capable of capturing peripheral light rays entering the eye at very large visual angles. As such, at least some of those rays (e.g., exemplary rays 21) miss the IOL and hence are only refracted by the cornea to form a second peripheral image (I2). Although this second peripheral image can expand the IOL user's peripheral vision, as noted above, it can also lead, in some cases, to dysphotopsia, e.g., due to the presence of a shadow region between the images. To alleviate this effect, in this case, the textured surface of the flange scatters some light rays (such as exemplary rays 23) incident thereon to such a shadow region, thereby ameliorating and preferably preventing the perception of peripheral visual artifacts.

While in the above exemplary IOL 10, the entire anterior surface of the flange 14 is textured, in other embodiments, only certain portions of that surface can be textured. For example, FIG. 3 schematically depicts an IOL 34 having a central optic 36 and a peripheral flange 38, where a portion 40 of the anterior surface of the flange, which receives peripheral light rays entering the eye at large visual angles from the temporal side, is textured.

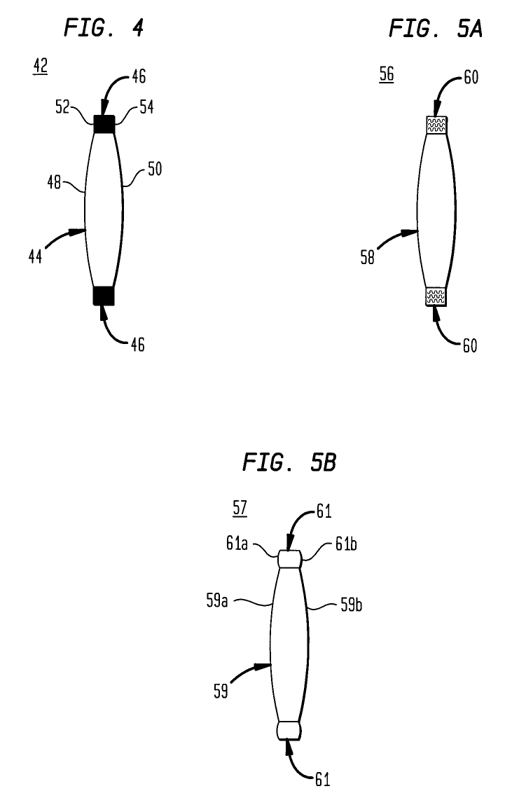

In other embodiments, the IOL's peripheral optical flange is opaque to visible radiation so as to inhibit dysphotopsia. By way of example, FIG. 4 schematically depicts an IOL 42 in accordance with such an embodiment that includes a central optic 44 that is surrounded by a peripheral flange 46. Though not shown, the IOL 42 can also include a plurality of fixation members (haptics) for facilitating its placement in a patient's eye. The central optic 44 includes an anterior surface 48 and a posterior surface 50 that cooperatively provide a desired optical power for imaging a field of view on the patient's retina. Further, the peripheral optical flange includes an anterior surface 52 and a posterior surface 54. Although in this embodiment, the flange's anterior and posterior surfaces are substantially flat, in other embodiments they can have curved profiles.

With continued reference to FIG. 4, the flange 46 is opaque to visible radiation so as to inhibit peripheral light rays entering the eye at large visual angles from reaching the retina, or to reduce the intensity of those rays. The term “opaque to visible radiation,” as used herein, refers to an opacity that would result in a reduction in the intensity of visible radiation, e.g., radiation with wavelengths in a range of about 380 nm to about 780 nm, by more than about 25%, or by more than about 40%, or by more than about 90%, or by more than about 95%, or by 100%. By way of example, in many embodiments, the intensity of incident radiation passing through the opaque flange is reduced by a factor greater than about 25% and more preferably by a factor greater than about 50%.

In some cases, the opacity of the flange is achieved by impregnating the biocompatible material of the flange with one or more dyes having absorption spectra in the visible wavelength regime. Some examples of such dyes are provided in U.S. Pat. Nos. 5,528,322 (entitled “Polymerizable Yellow Dyes And Their Use In Ophthalmic Lenses”), 5,470,932 (entitled “Polymerizable Yellow Dyes And Their Use In Ophthalmic Lenses”), 5,543,504 (entitled “Polymerizable Yellow Dyes And Their Use In Ophthalmic Lenses), and 5,662,707 (entitled “Polymerizable Yellow Dyes And Their Use In Ophthalmic Lenses), all of which are herein incorporated by reference. Further, while in this embodiment the entire peripheral extension is opaque, in other embodiments such opacity can be imparted to only portions of the peripheral extension, e.g., portions in proximity of the extension's anterior and/or posterior surfaces.

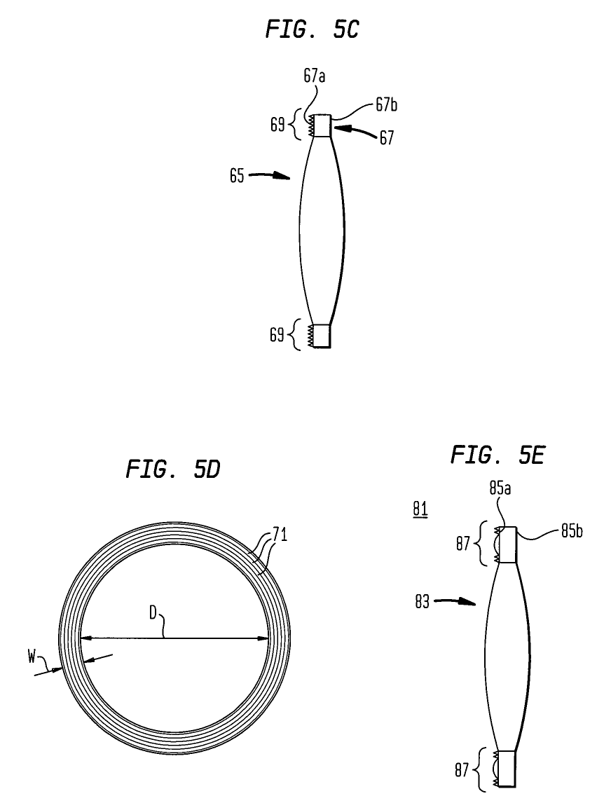

In other embodiments, the peripheral flange can be translucent so as to inhibit the peripheral light rays that enter the eye at large visual angles from generating a secondary peripheral image or to cause the diffusion of light passing therethrough such that a portion of the light reaches a shadow region between such a secondary peripheral image and a primary image formed by the IOL. By way of example, FIG. 5A schematically depicts an IOL 56 according to such an embodiment that includes a central optic 58 and a peripheral flange 60 that surrounds the optic. The peripheral flange is translucent to visible radiation. As such, it allows the peripheral light rays to pass therethrough, but diffusely. This can prevent the formation of a secondary image, or can cause some of the light to be incident on a reduced light intensity retinal region between a second peripheral image and the IOL's primary image, thereby preventing or at least ameliorate dysphotopsia. By way of example, the translucent flange can be formed by incorporating scattering centers in a biocompatible transparent polymeric material. In some cases, the peripheral flange can be made translucent by creating surface undulations (or roughness) on at least a surface thereof with amplitudes in a range of about 0.2 microns to about 2 microns, and preferably, in a range of about 0.2 microns to about 0.4 microns.

In yet other embodiments, the peripheral flange can include one or more curved surfaces adapted to direct the peripheral rays entering the eye at large visual angles towards the periphery of an image formed by the central optic on the patient's retina to enhance the IOL user's peripheral vision while inhibiting dysphotopsia. By way of example, FIG. 5B schematically depicts an IOL 57 having a central optic 59 to which an optical flange 61 is coupled. The central optic 59 is in the form of a biconvex lens comprising an anterior surface 59a and a posterior surface 59b, though other shapes such as plano-convex or plano-concave are also possible. The curvatures of the anterior and the posterior surfaces are selected such that the central optic would provide a desired optical power, e.g., in a range of about −15 to about +40 D, for generating an image of a field of view. Though not shown, the IOL 57 can include haptics for secure implantation in the eye.

With continued reference to FIG. 5B, the peripheral flange is also formed of an anterior surface 61a and a posterior surface 61b, both of which are curved. In many embodiments, the curvatures of those surfaces are such that the flange would provide an optical power that is substantially the same as that of the central optic 59. In such embodiments, the flange would focus the peripheral light rays incident thereon onto the retina such that they would form, together with the rays focused by the central optic, a single image of a field of view.

In some other embodiments, the optical power provided by the flange can be less than that of the central optic. For example, the optical power of the flange can differ from that of the central optic by a factor in a range of about 25% to about 75%. By way of example, in some embodiments, the optical power of the flange is less than by about 50% than that of the optic. In some cases, the optical power of the flange can be less than that of the cornea and/or that of the combined cornea and the optic (e.g., by a factor in a range of about 25% to about 75% (e.g., about 50%)).

In some cases, the flange can include a diffractive structure for directing light incident thereon to a shadow region between a secondary peripheral image formed by peripheral light rays entering the eye that miss the IOL and an image formed by the IOL. By way of example, FIG. 5C schematically depicts an IOL 63 having a central optic 65 and a peripheral flange 67, which has an anterior surface 67a and a posterior surface 67b, that surrounds the optic. A diffractive structure 69 is disposed on the anterior surface of the flange. The diffractive structure 69 is formed of a plurality of diffractive zones 71, each of which is separated from an adjacent zone by a step. In this embodiment, the step heights are uniform—although non-uniform heights are also possible in other embodiments—and can be represented by the following relationship:

$$Step height = \frac{λ}{a ( {n}_{2} - {n}_{1} )}$$

wherein,

λ denotes a design wavelength (e.g., 550 nm);

a denotes a parameter that can be adjusted to control diffraction efficiency associated with various orders, e.g., a can be selected to be 1,

n₂ denotes the index of refraction of the optic, and

n₁ denotes the refractive index of a medium in which the lens is placed.

In use, the diffractive structure 69 can direct at least some of the light rays incident thereon to a shadow region between a secondary peripheral image and an image formed by the IOL. In some implementations, the diffractive structure provides an optical power that is less than an optical power of the optic (e.g., by a factor in a range of about 25% to about 75%). As in many embodiments the diffractive structure 60 receives off-axis peripheral light rays, it can be characterized as having an effective optical power for bending such peripheral rays (e.g., rays entering the eye at visual angles in a range of about 50 degrees to about 80 degrees) so that they would reach the shadow region of the retina between an image formed by the optic and one formed by rays entering the eye that miss the IOL.

In some embodiments, the flange includes a Fresnel lens for directing light to the retinal shadow region. By way of example, FIG. 5E schematically depicts an IOL 81 according to such an embodiment, which includes a central optic 83 surrounded by a peripheral flange 85, which has an anterior surface 85a and a posterior surface 85b. A Fresnel lens 87 is disposed on an anterior surface and is adapted to direct light rays incident thereon to the retinal shadow region. To this end, in many embodiments, the Fresnel lens has an optical power less than the optical power of the cornea alone and/or the optical power of the cornea and the IOL's optic. For example, the optical power of the Fresnel lens can be about one-half of the optical power of the cornea alone and/or that of the cornea and the IOL's optic.

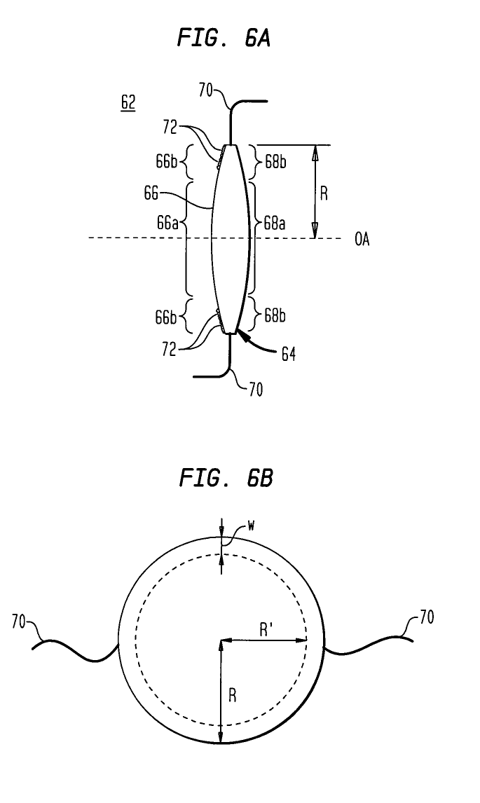

In other embodiments, rather than using a central optic and a separate peripheral flange, the IOL includes optical surfaces having a central portion that can function as a focusing surface for generating an image of a field of view and a peripheral portion that is adapted to inhibit dysphotopsia, e.g., by inhibiting the formation of a secondary image by peripheral light rays entering the eye at large visual angles or directing light into the shadow region. By way of example, FIGS. 6A and 6B schematically depict an IOL 62 in accordance with such an embodiment that includes an optic 64 having an anterior surface 66 and a posterior surface 68 disposed about an optical axis OA. The optic 64 can have a radial extension R relative to the optical axis in a range of about 2 mm to about 4.5 mm, and preferably in a range of about 2.5 mm to about 3.5 mm. The anterior and posterior surfaces can be characterized, respectively, as having central portions 66a and 68a that cooperatively form an image of a field of view, once IOL is implanted in a patient's eye, and peripheral portions 66b and 68b, which inhibit dysphotopsia, e.g., by preventing the formation of a secondary image. The central portions 66a and 68a can have a radius relative to the optical axis in a range of about 2 mm to about 3.5 mm and the peripheral portions 66b and 68b can have a width (w) in a range of about 0.5 mm to about 1 mm. Similar to the previous embodiments, the IOL 62 can include a pair of fixation members (haptics) 70 that facilitate its placement in the eye.

In this embodiment, the peripheral portion 66b of the anterior surface 66 includes a plurality of surface undulations 72 that cause scattering of light incident thereon. In other words, the peripheral portion of the anterior surface is textured. In many cases, the undulations have physical surface amplitudes in a range of about 0.2 microns to about 2 microns.

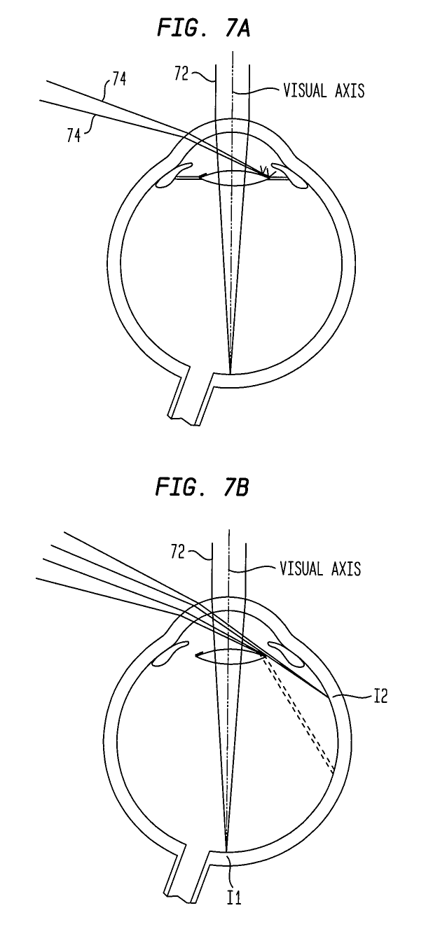

As shown schematically in FIG. 7A, in some implementations, once the IOL 62 is implanted in a patient's eye, the central portions of the anterior and the posterior surfaces form an image of a field of view, e.g., by focusing exemplary rays 72 onto the retina. The peripheral portion 66b of the IOL's anterior surface, however, receives peripheral light rays (such as rays 74) entering the eye at large visual angles, e.g., at angles greater than about 50 degrees, and causes the scattering of those rays. Such scattering inhibits those peripheral light rays from forming a secondary image that would lead to the perception of a dark shadow.

Alternatively, with reference to FIG. 7B, in some other implementations, the textured peripheral portion 66b of the IOL's anterior surface, rather than preventing the formation of a second peripheral image, directs some of the light rays incident thereon to a shadow region between such a secondary peripheral image (I2) and a primary image (I1) formed by the IOL.

While in this embodiment the peripheral portion of the anterior surface is textured, in other embodiments, the peripheral portion of the posterior surface, or the peripheral portions of both surfaces can be textured—though confining the texturing to the peripheral portion of the anterior surface is preferable because it can in some cases lower the risk of posterior capsular opacification (PCO).

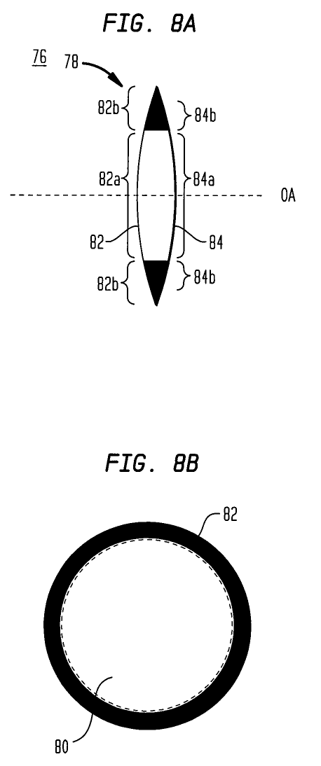

With reference to FIGS. 8A and 8B, in another embodiment, an IOL 76 includes an optic 78 disposed about an optical axis OA, where the optic includes a central portion 80 that is surrounded by a peripheral portion 82. More specifically, the IOL 76 includes an anterior surface 82 and a posterior surface 84, each of which extends from a central portion (portions 82a and 84a corresponding, respectively, to surfaces 82 and 84) to a peripheral portion (portions 82b and 84b corresponding, respectively, to surface 82 and 84). The optic 78 has a radius in a range of about 2 mm to about 4.5 mm, with the central portion having a radius in a range of about 2 mm to about 3.5 mm and the peripheral portion having a width in a range of about 0.5 mm to about 1 mm. In many embodiments, the opaque peripheral portion can be formed by impregnating the biocompatible polymeric material forming the lens with one or more suitable dye(s).

In this embodiment, the peripheral portion 82 is opaque to the visible radiation. Once the IOL 76 is implanted in a patient's eye, the central portion of the optic forms an image of a field of view. A plurality of peripheral light rays entering the eye at large visual angles are, however, incident on the peripheral portion of the IOL 76. As the peripheral portion is opaque, a substantial number of such peripheral rays (and in some cases all of them) do not reach the retina, thereby inhibiting the formation of a secondary peripheral image or causing a substantial attenuation of its intensity. By way of example, the peripheral portion can reduce the intensity of light rays passing therethrough by at least about 25%, or by at least about 40%, or by at least about 90%, or by at least about 95%, or by 100%.

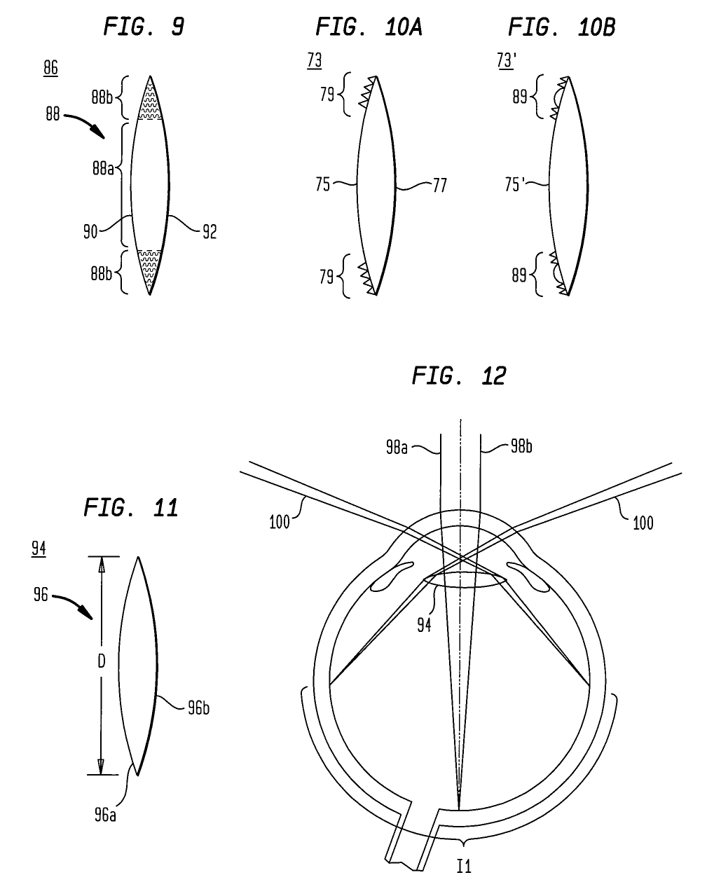

FIG. 9 schematically depicts an IOL 86 in accordance with another embodiment of the invention that includes an optic 88 formed of an anterior surface 90 and a posterior surface 92. The optic 88 includes a central portion 88a, which is adapted to form an image of a field of view, and a translucent peripheral portion 88b, which is adapted to inhibit dysphotopsia. In many cases, the central portion of the optic has a radius in a range of about 2 mm to about 3.5 mm and the translucent annular portion has a width (w) in a range of about 0.5 mm to about 1 mm. In use, the IOL's translucent portion receives the light rays entering the eye at large visual angles and inhibits those rays from forming a secondary peripheral image on the retina. Alternatively, in some implementations, rather than preventing the formation of a second peripheral image, the translucent portion directs at least some light rays incident thereon onto a shadow region between such a secondary peripheral image and the IOL's primary image to inhibit dysphotopsia.

With reference to FIG. 10A, in some embodiments, an IOL 73 can include an anterior surface 75 and a posterior surface 77, and a diffractive structure 79 disposed on a peripheral portion of its anterior surface (or in other implementations on a peripheral portion of the posterior surface) that can direct some of light rays incident thereon to a shadow region between a secondary peripheral image and an image formed by the IOL. By way of example, the parameters of the diffractive structure can be selected in a manner discussed above in connection with the aforementioned IOL 63. With reference to FIG. 10B, in some implementations, a Fresnel lens 89 is disposed on a peripheral portion of an anterior surface 75′ of an IOL 73′ to direct light incident thereon to the retinal shadow region. In some cases, the optical power of such a Fresnel lens is less than (e.g., about one-half) of that of the cornea alone and/or that of the combined cornea and the IOL.

In other embodiments, an IOL is provided that includes a focusing optic that is sufficiently large to inhibit dysphotopsia. By way of example, FIG. 11 schematically depicts an IOL 94 in accordance with such an embodiment, which includes an optic 96 having a diameter greater than about 6.5 mm—preferably in a range of about 6.5 mm to about 8 mm. The optic is formed of an anterior surface 96a and a posterior surface 96b, which cooperatively provide an image of a field of view. In many embodiments, the anterior and the posterior surfaces cooperatively provide an optical power in a range of about −15 D to about 40 D.

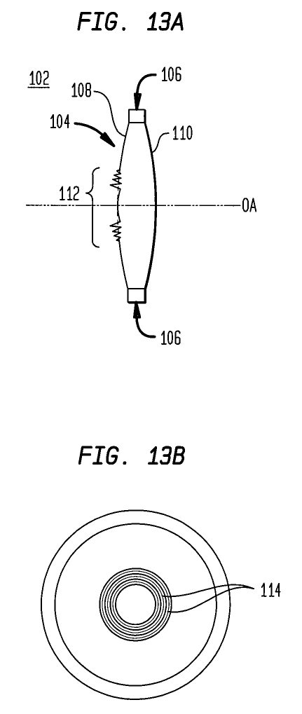

With reference to FIG. 12, once the IOL 94 is implanted in a patient's eye, the optic 96 focuses not only central rays (such as rays 98a and 98b) but also the peripheral rays (such as exemplary rays 100) entering the eye at large visual angles, e.g., at angles in a range of about 50 degrees to about 80 degrees, to form a single image I1 of a field of view. In other words, the optic receives the peripheral light rays and ensures that they are focused so as to generate the peripheral portion of a single image formed by the IOL.

In some implementations, the IOL 94 can have at least one aspheric surface characterized, e.g., by a conic constant in a range of about −10 to about −100, or in a range of about −15 to about −25. Further, in some cases, at least one surface of the IOL 94 can have a toric profile (i.e., a profile characterized by two different optical powers along two orthogonal surface directions). Additional teachings regarding the use of aspheric and/or toric surfaces in IOLs, such as various embodiments discussed herein, can be found in U.S. patent application Ser. No. 11/000,728 entitled “Contrast-Enhancing Aspheric Intraocular Lens,” filed on Dec. 1, 2004 and published as Publication No. 2006/0116763, which is herein incorporated by reference in its entirety.

Although in the above embodiments, the IOL provides a single optical power, in other embodiments, a multi-focal IOL can be provided, e.g., by utilizing a diffractive structure so as to provide both a far-focus optical power as well as a near-focus power. By way of example, such a diffractive structure can be disposed on an anterior surface (or a posterior surface or both surfaces) of the optic of the IOL corresponding to any of the aforementioned embodiments. For example, with reference to FIGS. 13A and 13B, an IOL 102 in accordance with one such embodiment includes a central optic 104 surrounded by a peripheral flange 106, which are disposed about an optical axis OA. The central optic includes an anterior surface 108 and a posterior surface 110. Once the IOL is implanted in a patient's eye, the central optic forms an image of a field of view on the patient's retina and the peripheral flange inhibits dysphotopsia. To this end, in some embodiments, the peripheral flange causes scattering of peripheral light rays entering the eye at large visual angles while in other embodiments the peripheral flange can be opaque or translucent to inhibit formation of a secondary image by those peripheral light rays. The curvatures of the anterior and posterior surfaces of the optic are selected such that the IOL would provide a desired far-focus optical power, e.g., in a range of about −15 D to about 34 D.

With continued reference to FIGS. 13A and 13B, A diffractive structure 108 that is disposed on the anterior surface provides a near focus optical power, e.g., in a range of about 1 D to about 4 D. In this embodiment, the diffractive structure 108 includes a plurality of diffractive zones 110 that are separated from one another by a plurality of steps that exhibit a decreasing height as a function of increasing distance from the optical axis OA—though in other embodiments the step heights can be uniform. In other words, in this embodiment, the step heights at the boundaries of the diffractive zones are “apodized” so as to modify the fraction of optical energy diffracted into the near and far foci as a function of aperture size (e.g., as the aperture size increases, more of the light energy is diffracted into the far focus). By way of example, the step height at each zone boundary can be defined in accordance with the following relation:

$$Step height = \frac{λ}{a ( {n}_{2} - {n}_{1} )} {f}_{apodize} \qquad Equation ( 1 )$$

wherein

λ denotes a design wavelength (e.g., 550 nm),

a denotes a parameter that can be adjusted to control diffraction efficiency associated with various orders, e.g., a can be selected to be 1.9;

n₂ denotes the index of refraction of the optic,

n₁ denotes the refractive index of a medium in which the lens is placed, and

ƒ_{apodize} represents a scaling function whose value decreases as a function of increasing radial distance from the intersection of the optical axis with the anterior surface of the lens. By way of example, the scaling function ƒ_{apodize} can be defined by the following relation:

$${f}_{apodize} = 1 - {( \frac{{r}_{i}}{{r}_{out}} )}^{3} . \qquad Equation ( 2 )$$

wherein

rᵢ denotes the radial distance of the i^{th} zone,

rₒᵤₜ denotes the outer radius of the last bifocal diffractive zone. Other apodization scaling functions can also be employed, such as those disclosed in a co-pending patent application entitled “Apodized Aspheric Diffractive Lenses,” filed Dec. 1, 2004 and having a Ser. No. 11/000,770, which is herein incorporated by reference. In addition, further teachings regarding apodized diffractive lenses can be found in U.S. Pat. No. 5,688,142 entitled “Diffractive Multifocal Ophthalmic Lens,” which is herein incorporated by reference

In this exemplary embodiment, the diffractive zones are in the form of annular regions, where the radial location of a zone boundary (rᵢ) is defined in accordance with the following relation:

rᵢ²=(2i+1)λƒ Equation (3)

wherein

i denotes the zone number (i=0 denotes the central zone),

rᵢ denotes the radial location of the ith zone,

λ denotes the design wavelength, and

ƒ denotes an add power.

A variety of IOL fabrication techniques known in the art, such as injection molding, can be employed to form IOLs according to the teachings of the invention.

Those having ordinary skill in the art will appreciate that various changes can be made to the above embodiments without departing from the scope of the invention.

What is claimed is:

1. An intraocular lens (IOL), comprising
a central optic,
a peripheral optical flange surrounding said optic,
wherein the central optic forms an image of a field of view on the retina of a patient's eye in which the IOL is implanted and said peripheral flange inhibits the perception of visual artifacts in a peripheral visual field of the patient.

2. The IOL of claim 1, wherein said peripheral flange inhibits the formation of a secondary peripheral image by peripheral light rays entering the eye that miss the IOL.

3. The IOL of claim 1, wherein said peripheral flange directs some light rays to a shadow region between an image formed by the IOL and a secondary image formed by peripheral light rays entering the eye that miss the IOL.

4. The IOL of claim 1, wherein said central optic has a radius in a range of about 2 mm to about 3.5 mm and said peripheral flange has a width in a range of about 0.5 mm to about 1 mm.

5. The IOL of claim 1, wherein said peripheral flange includes at least one textured surface.

6. The IOL of claim 5, wherein said textured surface includes a plurality of undulations with physical surface amplitudes in a range of about 0.2 microns to about 2 microns.

7. The IOL of claim 1, wherein said peripheral flange comprises an anterior surface and a posterior surface, wherein said textured surface forms the anterior surface.

8. The IOL of claim 6, wherein said textured surface is adapted to receive peripheral light rays entering the eye at large visual angles and to cause scattering thereof so as to prevent those rays from forming a secondary image.

9. The IOL of claim 6, wherein said textured surface is adapted to scatter at least some light rays incident thereon onto a shadow region between an image formed by the IOL and a secondary peripheral image formed by peripheral light rays entering the eye that miss the IOL.

10. The IOL of claim 1, wherein said flange is opaque to visible radiation.

11. The IOL of claim 10, wherein said opaque flange is adapted to receive peripheral light rays entering the eye at large visual angles and to inhibit those rays from forming a secondary image on the retina.

12. The IOL of claim 10, wherein said opaque flange is adapted to receive peripheral light rays entering the eye at large visual angles and to cause attenuation of an intensity of a secondary peripheral image formed by those rays.

13. The IOL of claim 1, wherein said flange is translucent to visible radiation.

14. The IOL of claim 13, wherein said translucent flange is adapted to receive peripheral light rays entering the eye at large visual angles and to inhibit those rays from forming a secondary image on the retina that would cause perception of a dark shadow in the patient's visual field.

15. The IOL of claim 13, wherein said translucent flange causes diffusion of at least some light rays incident thereon onto a shadow region between an image formed by the IOL and a secondary peripheral image formed by light rays entering the eye that miss the IOL.

16. The IOL of claim 1, further comprising a diffractive structure disposed on a surface of said flange.

17. The IOL of claim 1, wherein said diffractive structure provides an optical power less than an optical power of said optic.

18. The IOL of claim 1, wherein said diffractive structure provides an optical power less tan an optical power of the cornea.

19. The IOL of claim 1, wherein said diffractive structure provides an optical power less than a combined optical power of the cornea and the optic.

20. The IOL of claim 1, wherein said flange includes one or more curved surfaces for providing a refractive optical power.

21. The IOL of claim 20, wherein said optical power of the flange is less than an optical power of said optic by a factor in a range of about 25% to about 75%.

22. The IOL of claim 20, wherein said optical power of flange is less than any of the optical power of the cornea or the combined optical power of the cornea and that of the optic.

23. The IOL of claim 1, wherein said optic is foldable so as to allow its insertion into the eye.

24. The IOL of claim 1, further comprising a diffractive structure disposed on a surface of said central optic.

25. The IOL of claim 24, wherein said diffractive structure provides a near-focus optical power in a range of about 1 D to about 4 D.

26. The IOL of claim 24, wherein said optic comprises an anterior optical surface and a posterior optical surface, and wherein said diffractive structure is disposed on said anterior surface.

27. An intraocular lens (IOL), comprising
an optic comprising an anterior surface and a posterior surface,
said optic being characterized as having a central portion extending to a peripheral portion,
wherein said optic forms an image of a field of view on the retina of a patient's eye in which the IOL is implanted and said peripheral portion is adapted to inhibit the perception of visual artifacts in a peripheral visual field of the patient.

28. The IOL of claim 27, wherein said optic has a diameter in a range of about 4 mm to about 9 mm.

29. The IOL of claim 27, wherein said peripheral portion of the optic includes a textured region adapted to scatter light rays incident thereon.

30. The IOL of claim 29, wherein a peripheral portion of said anterior surface of the optic contains said textured region.

31. The IOL of claim 27, wherein said peripheral portion is opaque to visible radiation.

32. The IOL of claim 31, wherein said opaque peripheral portion is adapted to receive peripheral light rays entering the eye at large visual angles and to inhibit those rays from forming a secondary image on the retina.

33. The IOL of claim 31, wherein said opaque peripheral portion is adapted to receive peripheral light rays entering the eye at large visual angles and to cause attenuation in intensity of a secondary peripheral image formed by those rays.

34. The IOL of claim 27, wherein said peripheral portion is translucent to visible radiation.

35. The IOL of claim 34, wherein said translucent peripheral portion is adapted to receive peripheral light rays entering the eye at large visual angles and to inhibit those rays from forming a secondary image on the retina.

36. The IOL of claim 34, wherein said translucent portion causes diffusion of at least some light rays incident thereon onto a shadow region between an image formed by the IOL and a secondary peripheral image formed by peripheral light rays entering the eye that miss the IOL.

37. The IOL of claim 27, wherein said peripheral portion provides focusing of light incident thereon such that it forms together with said central portion a single image of a field of view.

38. The IOL of claim 27, further comprising a diffractive structure disposed on at least one of said anterior or posterior surfaces.

39. The IOL of claim 38, wherein said diffractive structure provides a near-focus optical power in a range of about 1 D to about 4 D.

40. The IOL of claim 27, further comprising a Fresnel lens disposed on a surface of said peripheral portion.

41. The IOL of claim 40, wherein said Fresnel lens provides an optical power less than that of the eye's cornea.

42. The IOL of claim 40, wherein said Fresnel lens provides an optical power less than a combined power of the cornea and that of the optic.

43. A method of correcting vision, comprising
providing an IOL having a central optic and a peripheral flange surrounding said optic, wherein said optic is adapted to form an image of a field of view and said flange is adapted to inhibit dysphotopsia,
implanting said IOL in a patient's eye.

44. A method of inhibiting dyspohotopsia in a visual field of a patient's eye in which an IOL is implanted, comprising
providing the IOL with a peripheral portion adapted to receive peripheral light rays entering the eye at large visual angles and inhibiting those rays from causing dysphotopsia.
